(19) United States
(12) Patent Application Publication
(10) Pub. No.: US 2008/0183199 A1
(43) Pub. Date: Jul. 31, 2008

(54) Membrane Scraper

(76) Inventor: Jurg Attinger, Stein am Rhein (CH)

Correspondence Address: ALCON, IP LEGAL, TB4-8, 6201 SOUTH FREEWAY, FORT WORTH, TX, 76134, US

(21) Appl. No.: 11/668,607

(22) Filed: Jan. 30, 2007

(51) Int. Cl. A61F 9/00

(52) U.S. Cl. 606/161

(57) ABSTRACT

A probe having a handle and attached cannula. The cannula contains a snare or loop of metal or polymer wire that can be either of fixed length or adjustable/retractable. At least a portion of the loop is roughened, either by roughening the loop material itself, or by coating the loop with an abrasive material, such as diamond dust or similar abrasive material.

This invention relates to ophthalmic surgical equipment and more particularly to posterior segment ophthalmic surgical equipment.

### BACKGROUND OF THE INVENTION

A continuing challenge to vitreoretinal surgeons is the separation of proliferative membranes from the neurosensory retina without injury or harm to the neurosensory retina during treatment for proliferative vitreoretinal disorders. For such a treatment, the removal of proliferative membranes from the surface of the retina is required in a wide variety of pathologic conditions and surgical situations. Various intraocular picks and intraocular forceps have been previously used for the removal of proliferative membranes.

However, prior art proliferative membrane removal techniques using conventional instruments carries the risk of causing damage to the retina at all times. In addition, “immature proliferative membranes”, seen in some proliferative vitreoretinal disorders, may be friable, difficult to peel off as films, and often cannot be sufficiently removed from the surface of the retina. The unremoved or remaining proliferative membranes can be the source of subsequent reproliferation over time.

One prior art device, disclosed in U.S. Pat. No. 5,921,998 (Tano, et al.) and commercially marketed as the “Tano Scraper” consists of a rigid cannula having a relatively flexible tube attached to its distal end. The tube is coated with an abrasive, such as diamond dust. The distal end of the cannula is rubbed over the tissue to be removed, providing a gentle scrubbing or sanding action. As the flexible tube is opaque, it can be difficult to see the surface being rubbed.

Accordingly, a need continues to exist for a membrane scraper having increased visibility.

### BRIEF SUMMARY OF THE INVENTION

The present invention improves upon prior art by providing a probe having a handle and attached cannula. The cannula contains a snare or loop of metal or polymer wire that can be either of fixed length or adjustable/retractable. At least a portion of the loop is roughened, either by roughening the loop material itself, or by coating the loop with an abrasive material, such as diamond dust or similar abrasive material.

Accordingly, an objective of the present invention to provide an ophthalmic membrane scraper.

Another objective of the present invention is to provide a membrane scraper having increases visibility of the surface being scraped.

Another objective of the present invention to provide an ophthalmic membrane scraper having a loop or snare.

A further objective of the present invention to provide an ophthalmic membrane scraper having a roughened loop or snare.

Other objectives, features and advantages of the present invention will become apparent with reference to the drawings, and the following description of the drawings and claims.

### BRIEF DESCRIPTION OF THE DRAWINGS

FIG. 1 is an enlarged perspective view of a first embodiment of the membrane scraper of the present invention.

FIG. 2 is an enlarged perspective view of one embodiment of the distal tip of the membrane scraper of the present invention, taken at circle 2 in FIG. 1.

FIG. 3 is an enlarged cross-sectional view of a second embodiment of the membrane scraper of the present invention.

FIG. 4 is an enlarged perspective view of a second embodiment of the distal tip of the membrane scraper of the present invention, taken at circle 4 in FIG. 3.

### DETAILED DESCRIPTION OF THE DRAWINGS

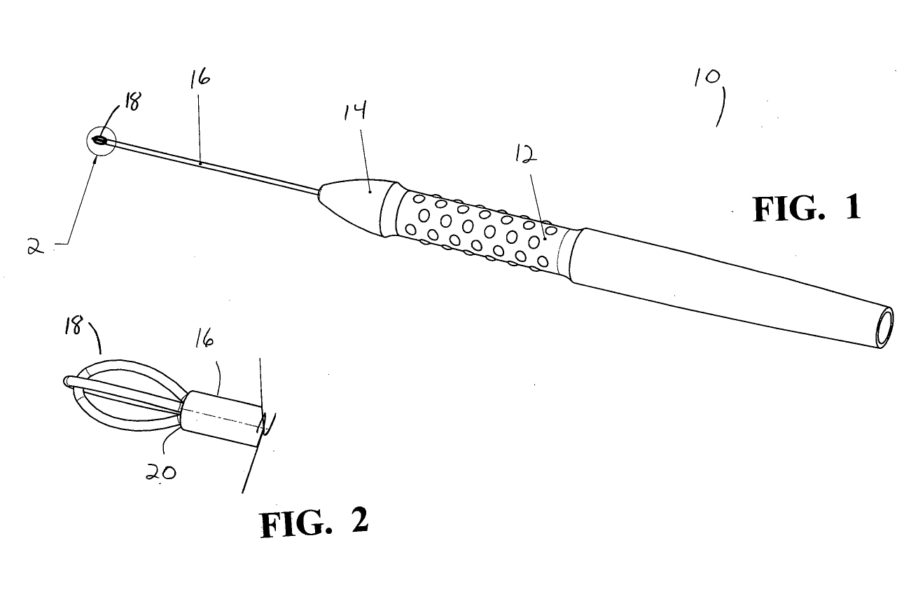

As best seen in FIG. 1, a first embodiment of membrane scraper 10 of the present invention generally includes handle 12, nosecone or hub 14, cannula 16 and loop or snare 18. Cannula 16 attaches to hub 14 and hub 14 attaches to handle 12 by any of a variety of know attachments methods, such as adhesive, ultrasonic welding, heat welding or crimping. Cannula 16 preferably is made from 20 ga., 23 ga. or 25 ga. or smaller stainless steel or titanium tubing, and hub 14 and handle 12 preferably or molded from thermoplastic or formed from stainless steel or titanium by methods well-known in the art. Loop 16 preferably is formed from stainless steel, titanium or polymer wire and attached to distal end 20 of cannula 16 by adhesive, welding or crimping. As best seen in FIGS. 2 and 4, membrane scraper 10 may contain a single loop 18 or a plurality of loops 18. Loop(s) 18 preferable have a roughened texture, which can be accomplished by roughened loop 18 directly (e.g., sandblasting or cutting serration into loop 18) or by applying a roughening coating to loop 18 (e.g., diamond or other mineral or metallic dust). Loop(s) 18 allow the surface being acted upon to be visualized more easily, which is especially important with delicate eye tissues.

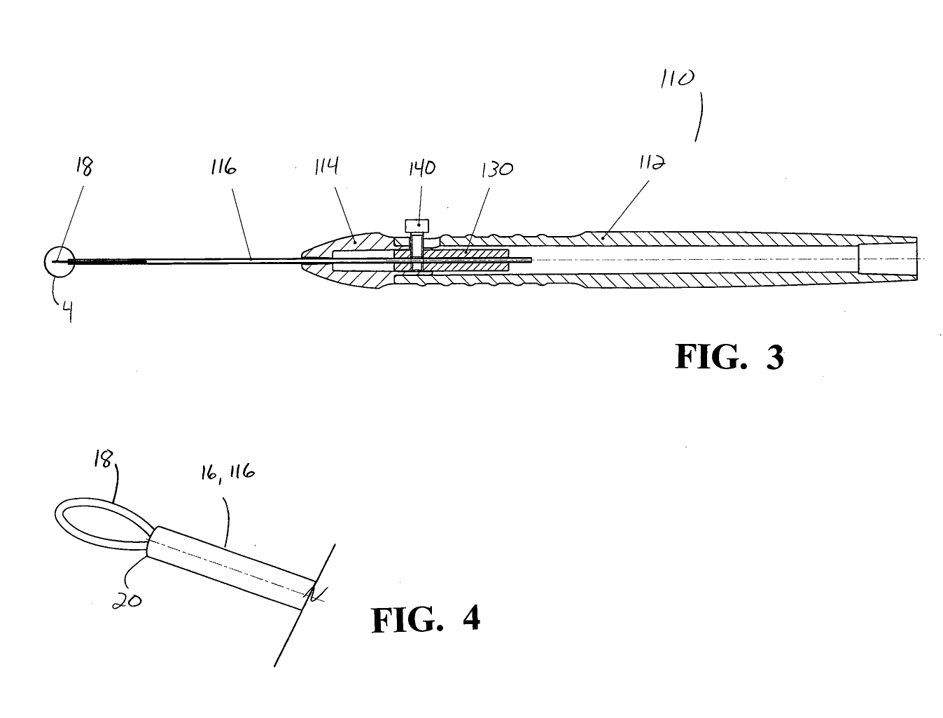

Alternatively, as best seen in FIG. 3, membrane scraper 110 may contain loop(s) 18 that extend through cannula 116 and be attached to sliding block 130 that reciprocated within hollow handle 112 and nosecone 114. Reciprocation of block 130 is achieved by manipulation of button or lever 140. Movement of button 140 forward (distally) causes loop(s) 18 to be pushed forward and extend out of distal end 20 of cannula 116, as shown in FIGS. 2 and 4. Movement of button 140 rearward (proximally) causes loop(s) 18 to be retracted within distal end 20 of cannula 116.

While certain embodiments of the present invention have been described above, these descriptions are given for purposes of illustration and explanation. Variations, changes, modifications and departures from the systems and methods disclosed above may be adopted without departure from the scope or spirit of the present invention.

I claim:

1. A membrane scraper, comprising:
a) a hollow handle having a nosecone;
b) a sliding block that reciprocates within the hollow handle;
b) a cannula attached to the nosecone; and
c) at least one wire loop extending through the cannula and attached to the sliding block so that movement of the sliding block causes the wire loop to reciprocate within the cannula.

2. The membrane scraper of claim 1 wherein the wire loop is roughened.

3. The membrane scraper of claim 1 wherein the wire loop further comprises a roughening coating.

4. The membrane scraper of claim 1 further comprising a plurality of wire loops.

5. The membrane scraper of claim 4 wherein the wire loops are roughened.

6. The membrane scraper of claim 4 wherein the wire loops further comprise a roughening coating.

7. A membrane scraper, comprising:
a) a handle having a nosecone;
b) a cannula attached to the nosecone; and
c) at least one wire loop attached to the cannula opposite the nosecone.

8. The membrane scraper of claim 7 wherein the wire loop is roughened.

9. The membrane scraper of claim 7 wherein the wire loop further comprises a roughening coating.

10. The membrane scraper of claim 7 further comprising a plurality of wire loops.

11. The membrane scraper of claim 10 wherein the wire loops are roughened.

12. The membrane scraper of claim 10 wherein the wire loops further comprise a roughening coating.
